(19) United States
(12) Patent Application Publication
(10) Pub. No.: US 2017/0258462 A1
(43) Pub. Date: Sep. 14, 2017

(54) Soft Tissue Retractor

(71) Applicant: FEHLING INSTRUMENTS GMBH & Co. KG, Karlstein (DE)

(72) Inventor: Guido Fehling, Karlstein (DE)

(21) Appl. No.: 15/084,779

(22) Filed: Mar. 30, 2016

(30) Foreign Application Priority Data
Mar. 10, 2016 (DE) 20 2016 101 334.8

(51) Int. Cl. A61B 17/02

(52) U.S. Cl. CPC A61B 17/0218; A61B 17/0293; A61B 2017/00867

(57) ABSTRACT

A soft tissue retractor for medical use, formed as a rectangular blade-like retractor element made from shape memory alloy.

The invention relates to a soft tissue retractor.

The use of soft tissue retractors serves for exposing surgical areas and for granting access for instruments, particularly in case of minimally invasive procedures. With the help of tissue retractors, the soft tissue is held back. Soft tissue retractors of prior art show two joint parts, articulated in reference to each other about a swivel axis, with a contact area being arranged at each joint part and allowing the distance of the contact areas from each other to be varied by pivoting the joint parts. The contact areas are made to contact the tissue to be retracted. Frequently, soft tissue retractors optionally allow the joint parts to be fixed at a certain relative position to each other in order to hold the contact areas at a desired distance. Such soft tissue retractors require a lot of space in the proximity of the surgical area, are frequently cumbersome in their handling, and obstruct the visibility and access by other instruments.

The objective of the invention is therefore to provide a soft tissue retractor, which requires only little space and can advantageously be handled easily.

The objective of the invention is attained in a soft tissue retractor showing the features of claim 1.

Advantageous configurations and implementations of the invention are disclosed in the dependent claims.

The soft tissue retractor according to the invention is characterized in that it is embodied essentially as a rectangular, blade-like retractor element made from a memory alloy. Such a retractor element can be coiled to a ring and then it can be inserted into the soft tissue surrounding the surgical area. Due to the fact that the retractor element is made from shape memory alloy, the retractor element tends to unroll in the coiled state. By this force the soft tissue surrounding the surgical area is held back. Such a retractor element requires only little space and particularly projects only slightly beyond the surface of the body, if at all. Such a retractor element can be inserted into the soft tissue via pliers, which can then be removed so that particularly no handles, joint parts, or the like come to rest in the surgical area, because only the blade-like retractor element is arranged in the soft tissue in the form of a coil with low wall thickness.

Advantageously the retractor element can be coiled about a transverse axis in order to allow a particularly low height of the retractor element to be inserted.

A preferred embodiment of the invention provides that the retractor element is made from an austenitic shape memory alloy. In an austenitic shape memory alloy the alloy is present in an austenitic phase, i.e. in a high-temperature phase in which it shows an austenitic structure. In this temperature range, which, for example, ranges from 10° C. to 80° C., the shape memory alloy shows so-called super-elastic behavior, which means that a reversible change of shape is possible caused by the impact of an external force. When the deforming force is removed, the material returns to its original shape without any temperature change being necessary.

The retractor element made from a nickel-titanium shape memory alloy, showing bio-compatible features, is particularly preferred.

According to an advantageous embodiment of the invention, the retractor element shows several openings, which are particularly arranged in a grid-like structure. The openings allow for the drainage of liquids, so that any stasis of fluids can be avoided. A grid-like structure can provide the blade-like retractor element with particular stability. For example, the openings may be formed in a rhomboidal fashion.

Advantageously, braces are arranged between the openings, which are positioned at an angle from 45° to 75° in reference to the longitudinal axis of the retractor element. Such an arrangement of the braces improves the stability of the retractor element.

The retractor element may show a length from 100 mm to 300 mm, preferably from 150 mm to 250 mm, particularly preferred, a length of approx. 200 mm. The retractor element may show a width from approximately 15 mm to 60 mm, preferably showing a width of approximately 25 mm or 40 mm or 55 mm. Advantageously the retractor element shows a thickness of less than 0.5 mm, for example a thickness of 0.45 mm or 0.35 mm or 0.25 mm or 0.12 mm. The optimal dimensions may be selected depending on the application of the soft tissue retractor.

An exemplary embodiment of the invention is explained in greater detail using the following figures, Wherein:

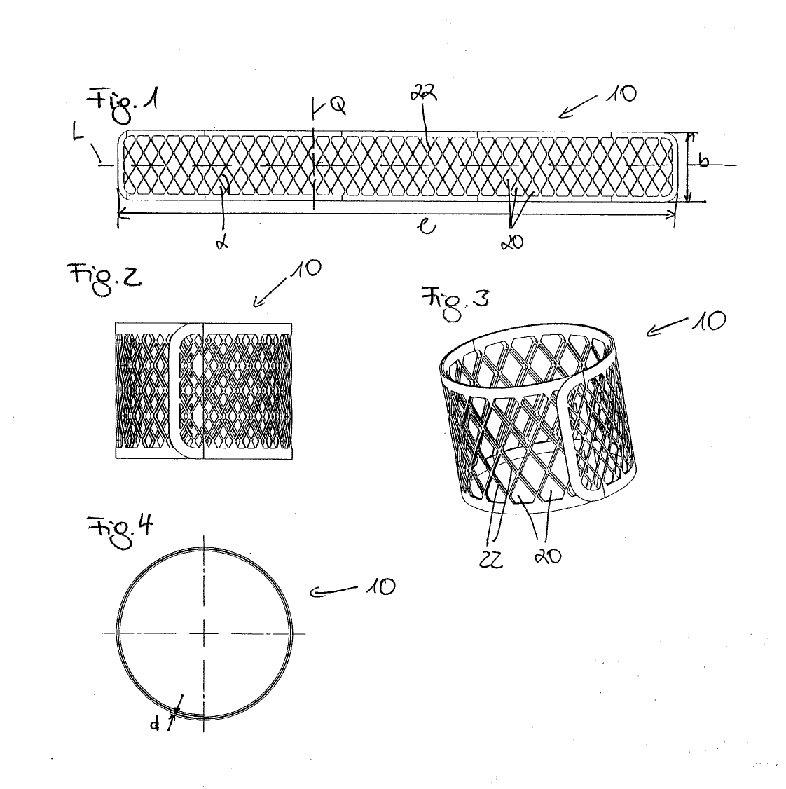

FIG. 1 shows a top view of an exemplary embodiment of a soft tissue retractor according to the invention,

FIG. 2 shows the soft tissue retractor according to FIG. 1 in the coiled state,

FIG. 3 shows a perspective view of the coiled soft tissue retractor according to FIG. 2, and

FIG. 4 shows a top view of the coiled soft tissue retractor according to FIG. 2.

In FIGS. 1 to 4, various illustrations are shown of the soft tissue retractor according to the invention, which is essentially embodied as a rectangular blade-like retractor element 10. The retractor element 10 shows a length l, a width b, and a thickness d. Furthermore, the retractor element 10 shows a longitudinal axis L, with the longitudinal axis L extending parallel in reference to the length l. A transverse axis Q extends perpendicular in reference to the longitudinal axis, however it does not necessarily intersect it and may for example extend parallel to the width b.

The retractor element 10 is made from shape memory alloy, for example austenitic shape memory alloy, particularly austenitic nickel-titanium shape memory alloy. Here, the retractor element 10 is particularly produced such that in the unstressed state the retractor element 10 shows the tendency to assume a blade-like form, particularly as shown in FIG. 1.

The retractor element 10 is embodied such that it can be coiled and can particularly be coiled to form a ring, in which the two longitudinal sides of the essentially rectangular blade-like retractor element 10 respectively form a ring and the two short ends of the retractor element 10 are pushed over the top of each other (particularly see FIGS. 2 to 4). Depending on the length 1 of the retractor element 10, the coiling may occur in several windings, which closely abut each other. As an example, in FIG. 4 the retractor element 10 is coiled to form approximately two windings. The coiling occurs particularly about a transverse axis Q of the retractor element 10.

In the coiled state, the retractor element 10 can be inserted into the soft tissue of a surgical area. By stressing the material, which tends to return to the blade-like form, the soft tissue is held back around the access to the surgical area. The retractor element 10 can be inserted by means of pliers into the surgical area. Here, particularly the ends of the retractor element 10 overlapping each other are held. As soon as the soft tissue retractor is placed at the desired location, the pliers can release the retractor element 10 and the pliers can then be removed from the surgical area.

The retractor element 10 may show several openings 20, which are arranged in the present exemplary embodiment in a grid-like structure and for example may be embodied in a rhomboidal fashion. Braces 22 may be arranged between the openings 20, which, for example, may be positioned inclined at an angle a from 45° to 75° in reference to the longitudinal axis L of the retractor element 10.

The retractor element may show a length from 100 mm to 300 mm, preferably from 150 mm to 250 mm, for example a length l of approx. 200 mm. Depending on the application of the retractor element 10, a retractor element 10 may be selected showing the desired length 1. The retractor element 10 preferably shows a width b from approx. 15 mm to 60 mm, for example a width b of approx. 25 mm or 40 mm or 55 mm. Depending on the application of the retractor element 10, the desired width b may be adjusted optimally. The retractor element 10 shows preferably a thickness d of less than 0.5 mm, for example a thickness of 0.45 mm or 0.35 mm or 0.25 mm or 0.12 mm. The desired thickness d may be selected depending on the application.

### LIST OF REFERENCE CHARACTERS

10 Retractor element
l Length
b Width
d Thickness
L Longitudinal axis
Q Lateral axis
20 Opening
22 Brace
α Angle

1. A soft tissue retractor, which is formed as an essentially rectangular blade-like retractor element (10) made from shape memory alloy.

2. A soft tissue retractor according to claim 1, characterized in that the retractor element (10) can particularly be coiled about a transverse axis (Q).

3. A soft tissue retractor according to one of the previous claims, characterized in that the retractor element (10) is made from austenitic shape memory alloy.

4. A soft tissue retractor according to one of the previous claims, characterized in that the retractor element (10) is made from a nickel-titanium shape memory alloy.

5. A soft tissue reactor according to one of the previous claims, characterized in that the retractor element (10) shows several openings (20), which are particularly arranged in a grid-like structure.

6. A soft tissue retractor according to claim 5, characterized in that the openings (20) are embodied in a rhomboidal fashion.

7. A soft tissue retractor according to claim 5 or 6, characterized in that braces (22) are arranged between the openings (20) which are arranged inclined at an angle (α) from 45° to 75° in reference to the longitudinal axis (l) of the retractor element (10).

8. A soft tissue retractor according to one of the previous claims, characterized in that the retractor element (10) shows a length (l) from 100 mm to 300 mm, preferably from 150 mm to 250 mm, particularly preferred shows a length (l) of approximately 200 mm.

9. A soft tissue retractor according to one of the previous claims, characterized in that the retractor element (10) shows a width (b) from approximately 15 mm to 60 mm, preferably a width (b) of approximately 25 mm or 40 mm or 55 mm.

10. A soft tissue retractor according to one of the previous claims, characterized in that the retractor element (10) shows a thickness (d) of less than 0.5 mm, preferably a thickness (d) of 0.45 mm or 0.35 mm or 0.25 mm or 0.12 mm.
